(19) United States
(12) Patent Application Publication
(10) Pub. No.: US 2026/0200842 A1
(43) Pub. Date: Jul. 16, 2026

(54) SULFUR-CONTAINING COMPOUND, SULFUR-CONTAINING POLYPHENYLENE OXIDE, FUNCTIONALIZED SULFUR-CONTAINING POLYPHENYLENE OXIDE, COMPOSITION, AND FILM

(71) Applicant: INDUSTRIAL TECHNOLOGY RESEARCH INSTITUTE, Hsinchu (TW)

(72) Inventors: Chung-Han YU, Changhua City (TW); Wei-Hua CHEN, Taoyuan City (TW); Po-Hsien HO, Taipei City (TW)

(73) Assignee: INDUSTRIAL TECHNOLOGY RESEARCH INSTITUTE, Hsinchu (TW)

(21) Appl. No.: 19/421,437

(22) Filed: Dec. 16, 2025

Related U.S. Application Data
(60) Provisional application No. 63/744,420, filed on Jan. 13, 2025.

(30) Foreign Application Priority Data
Nov. 26, 2025 (TW) 114146185

(51) Int. Cl. C07C 317/14; C08G 65/48; C08J 5/18

(52) U.S. Cl. CPC C07C 317/14; C08G 65/485; C08J 5/18; C08J 2371/12

(57) ABSTRACT

A functionalized sulfur-containing polyphenylene oxide is provided, which has a chemical structure of

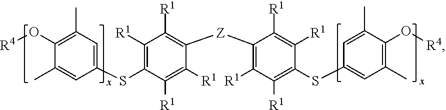

wherein each R¹ is independently H or F; Z is

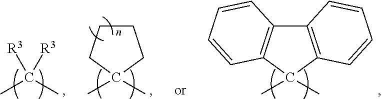

each R³ is independently H, F, C₁₋₆ linear alkyl group, C₁₋₆ fluorinated linear alkyl group, C₃₋₆ cycloalkyl group, C₃₋₆ fluorinated cycloalkyl group, C₃₋₆ branched alkyl group, or C₃₋₆ fluorinated branched alkyl group, and n is an integer of 0 to 10; each R⁴ is independently

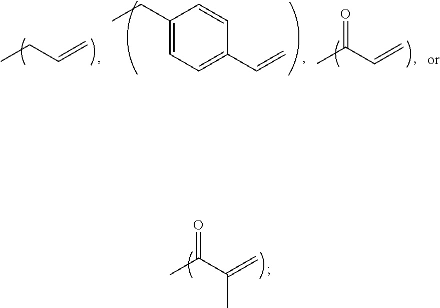

and each x is an independent integer of 1 to 60.

### CROSS REFERENCE TO RELATED APPLICATIONS

This application claims the benefit of U.S. Provisional Application No. 63/744,420 filed on Jan. 13, 2025, the entirety of which is incorporated by reference herein.

The present application is based on, and claims priority from, Taiwan Application Serial Number 114146185, filed on Nov. 26, 2025, the disclosure of which is hereby incorporated by reference herein in its entirety.

### TECHNICAL FIELD

The technical field relates to a sulfur-containing compound, a sulfur-containing polyphenylene oxide, a functionalized sulfur-containing polyphenylene oxide, a composition, and a film.

### BACKGROUND

Due to the advancements being made in semiconductor technology, as well as the strong demand for AI servers, low-orbit satellites, and automotive electronics, there are increasing requirements on the dielectric properties and mechanical characteristics of high-frequency circuit substrates.

Polyphenylene oxide (PPO) is an engineering plastic with excellent dielectric properties. PPO can be applied as a copper clad laminate substrate material in printed circuit boards (PCBs).

However, it is difficult for PPO to simultaneously have excellent dielectric properties and other related properties (e.g., dimensional stability and flame retardancy). If a large amount of phosphorous-containing flame retardant agent is added to enhance its flame retardancy, this may have an effect on the dielectric properties and dimensional stability of PPO in subsequent applications. As such, PPO simultaneously having dielectric properties and other related properties (e.g., dimensional stability and flame retardancy) is major development direction for the industries.

### SUMMARY

One embodiment of the disclosure provides a sulfur-containing compound, having a chemical structure of

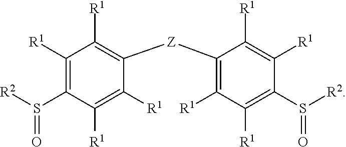

Each R¹ is independently H or F. Each R² is independently C₁₋₁₀ alkyl group or an aryl group substituted with a C₁₋₁₀ alkyl group. Z is

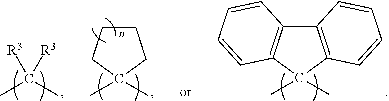

Each R³ is independently H, F, C₁₋₆ linear alkyl group, C₁₋₆ fluorinated linear alkyl group, C₃₋₆ cycloalkyl group, C₃₋₆ fluorinated cycloalkyl group, C₃₋₆ branched alkyl group, or C₃₋₆ fluorinated branched alkyl group. n is an integer of 0 to 10.

One embodiment of the disclosure provides a sulfur-containing polyphenylene oxide, having a chemical structure of

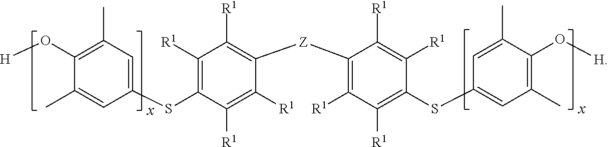

Each R¹ is independently H or F. Z is

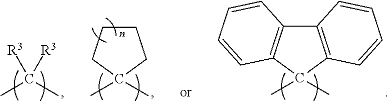

Each R³ is independently H, F, C₁₋₆ linear alkyl group, C₁₋₆ fluorinated linear alkyl group, C₃₋₆ cycloalkyl group, C₃₋₆ fluorinated cycloalkyl group, C₃₋₆ branched alkyl group, or C₃₋₆ fluorinated branched alkyl group. n is an integer of 0 to 10. Each x is an independent integer of 1 to 60.

One embodiment of the disclosure provides a functionalized sulfur-containing polyphenylene oxide, having a chemical structure of

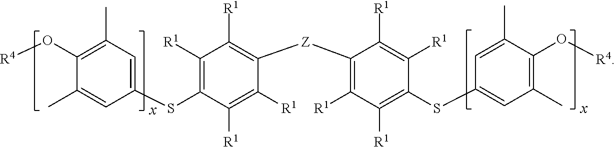

Each R¹ is independently H or F. Z is

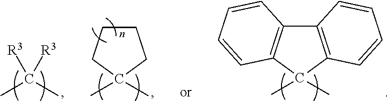

Each R³ is independently H, F, C₁₋₆ linear alkyl group, C₁₋₆ fluorinated linear alkyl group, C₃₋₆ cycloalkyl group, C₃₋₆ fluorinated cycloalkyl group, C₃₋₆ branched alkyl group, or C₃₋₆ fluorinated branched alkyl group. n is an integer of 0 to 10. Each R⁴ is independently

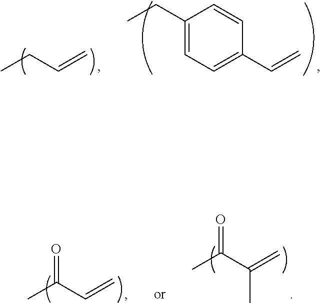

Each x is an independent integer of 1 to 60.

One embodiment of the disclosure provides a composition, including 100 parts by weight of the described functionalized sulfur-containing polyphenylene oxide; and 10 parts to 50 parts by weight of a bismaleimide resin.

One embodiment of the disclosure provides a film formed by curing the described composition.

A detailed description is given in the following embodiments.

### DETAILED DESCRIPTION

In the following detailed description, for purposes of explanation, numerous specific details are set forth in order to provide a thorough understanding of the disclosed embodiments. It will be apparent, however, that one or more embodiments may be practiced without these specific details.

One embodiment of the disclosure provides a sulfur-containing compound, having a chemical structure of

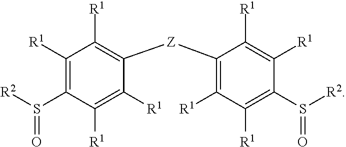

Each R¹ is independently H or F. Each R² is independently C₁₋₁₀ alkyl group or an aryl group substituted with a C₁₋₁₀ alkyl group. Z is

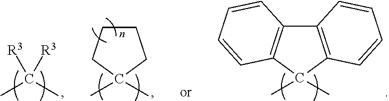

Each R³ is independently H, F, C₁₋₆ linear alkyl group, C₁₋₆ fluorinated linear alkyl group, C₃₋₆ cycloalkyl group, C₃₋₆ fluorinated cycloalkyl group, C₃₋₆ branched alkyl group, or C₃₋₆ fluorinated branched alkyl group. n is an integer of 0 to 10.

In some embodiments, the sulfur-containing compound is synthesized by following way. It should be understood that the following synthesis way is a possible synthesis way rather than the only synthesis way. One skilled in the art may select any available synthesis steps to synthesize the sulfur-containing compound. For example, ketone, thioanisole, and 3-mercaptopropionic acid can react as below:

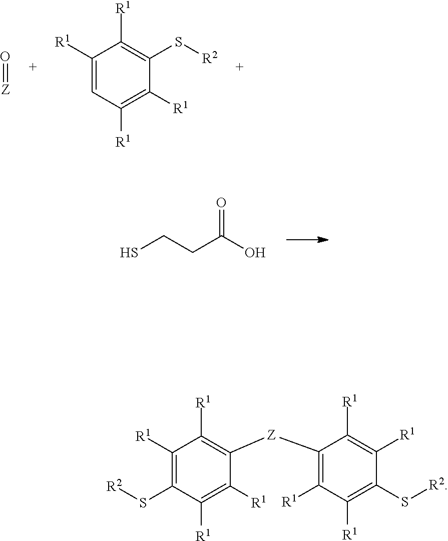

Subsequently, the sulfide groups are oxidized to form sulfoxide groups, as shown below:

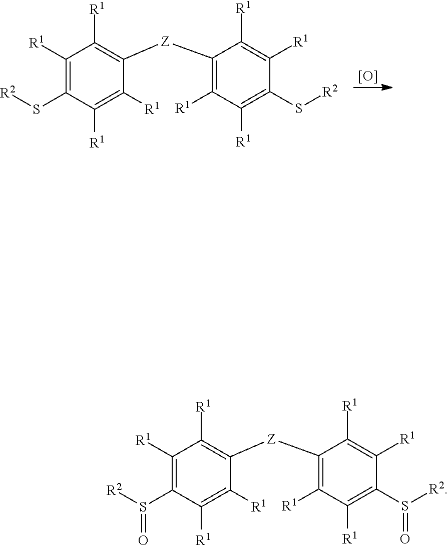

In some embodiments, the sulfur-containing compound has a chemical structure

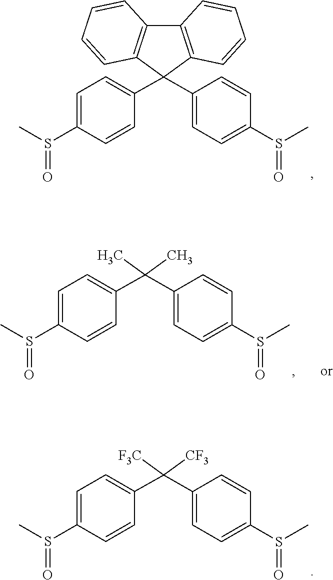

One embodiment of the disclosure provides a sulfur-containing polyphenylene oxide, having a chemical structure of

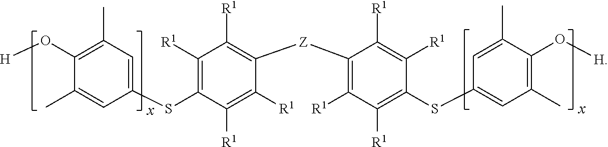

Each R¹ is independently H or F. Z is

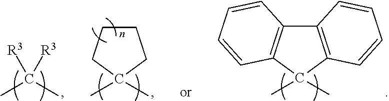

Each R³ is independently H, F, C₁₋₆ linear alkyl group, C₁₋₆ fluorinated linear alkyl group, C₃₋₆ cycloalkyl group, C₃₋₆ fluorinated cycloalkyl group, C₃₋₆ branched alkyl group, or C₃₋₆ fluorinated branched alkyl group. n is an integer of 0 to 10. Each x is an independent integer of 1 to 60. In some embodiments, each x is an independent integer of 1 to 35.

In some embodiments, the sulfur-containing polyphenylene oxide is synthesized by following way. It should be understood that the following synthesis way is a possible synthesis way rather than the only synthesis way. One skilled in the art may select any available synthesis steps to synthesize the sulfur-containing polyphenylene oxide. For example, the sulfur-containing compound, a polyphenylene oxide oligomer, and Eaton's reagent can react and then reflux to form the sulfur-containing polyphenylene oxide, as shown below:

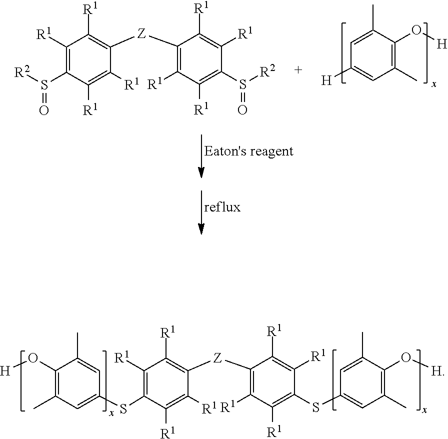

In some embodiments, the sulfur-containing polyphenylene oxide has a chemical structure

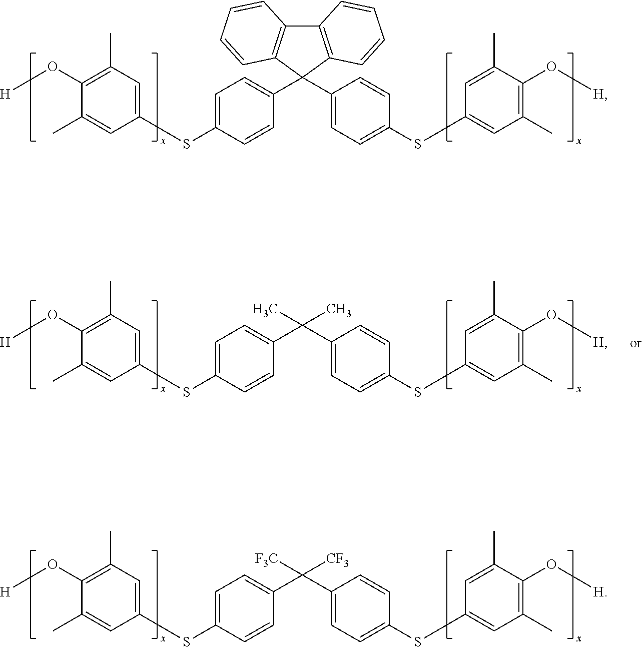

One embodiment of the disclosure provides a functionalized sulfur-containing polyphenylene oxide. having a chemical structure of

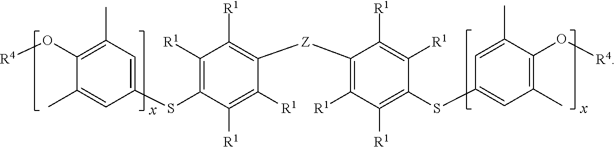

Each R¹ is independently H or F. Z is

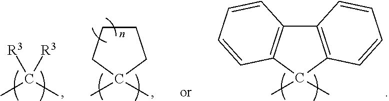

Each R³ is independently H, F, C₁₋₆ linear alkyl group, C₁₋₆ fluorinated linear alkyl group, C₃₋₆ cycloalkyl group, C₃₋₆ fluorinated cycloalkyl group, C₃₋₆ branched alkyl group, or C₃₋₆ fluorinated branched alkyl group. n is an integer of 0 to 10. Each R⁴ is independently

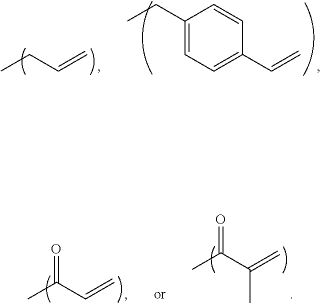

Each x is an independent integer of 1 to 60. In some embodiments, each x is an independent integer of 1 to 35.

In some embodiments, the functionalized sulfur-containing polyphenylene oxide is synthesized by following way. It should be understood that the following synthesis way is a possible synthesis way rather than the only synthesis way. One skilled in the art may select any available synthesis steps to synthesize the functionalized sulfur-containing polyphenylene oxide. For example, the sulfur-containing polyphenylene oxide and acrylic acid-based compound or an anhydride thereof can progress an esterification, as shown below:

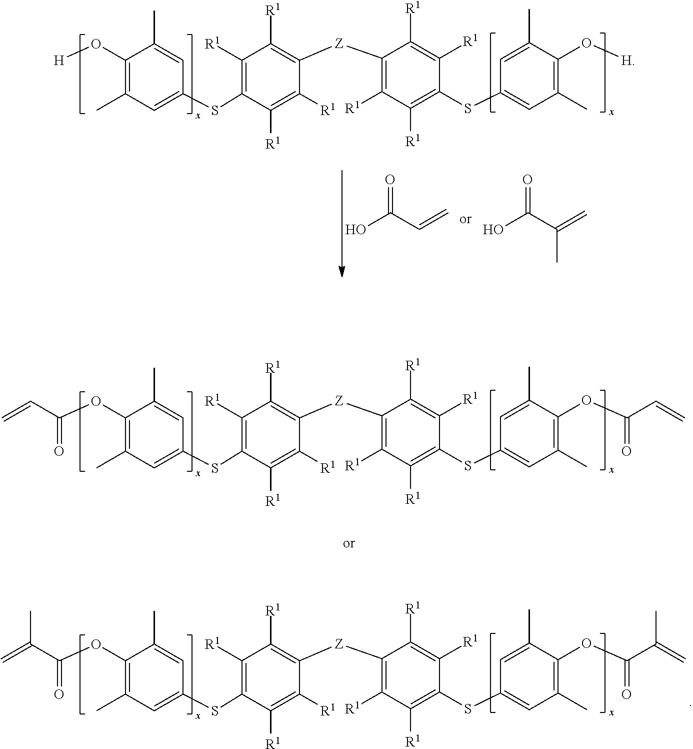

On the other hand, the sulfur-containing polyphenylene oxide and R⁴—X can (R⁴ is

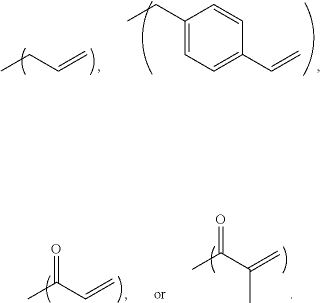

and X is Cl, Br, or I) progress an substitution reaction, as shown below:

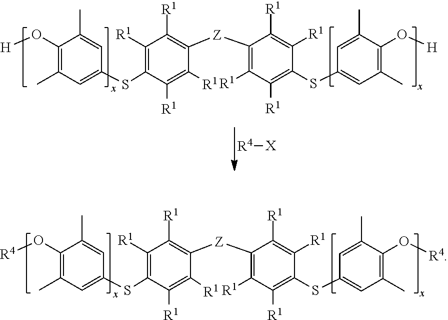

In some embodiments, the functionalized sulfur-containing polyphenylene oxide has a chemical structure of

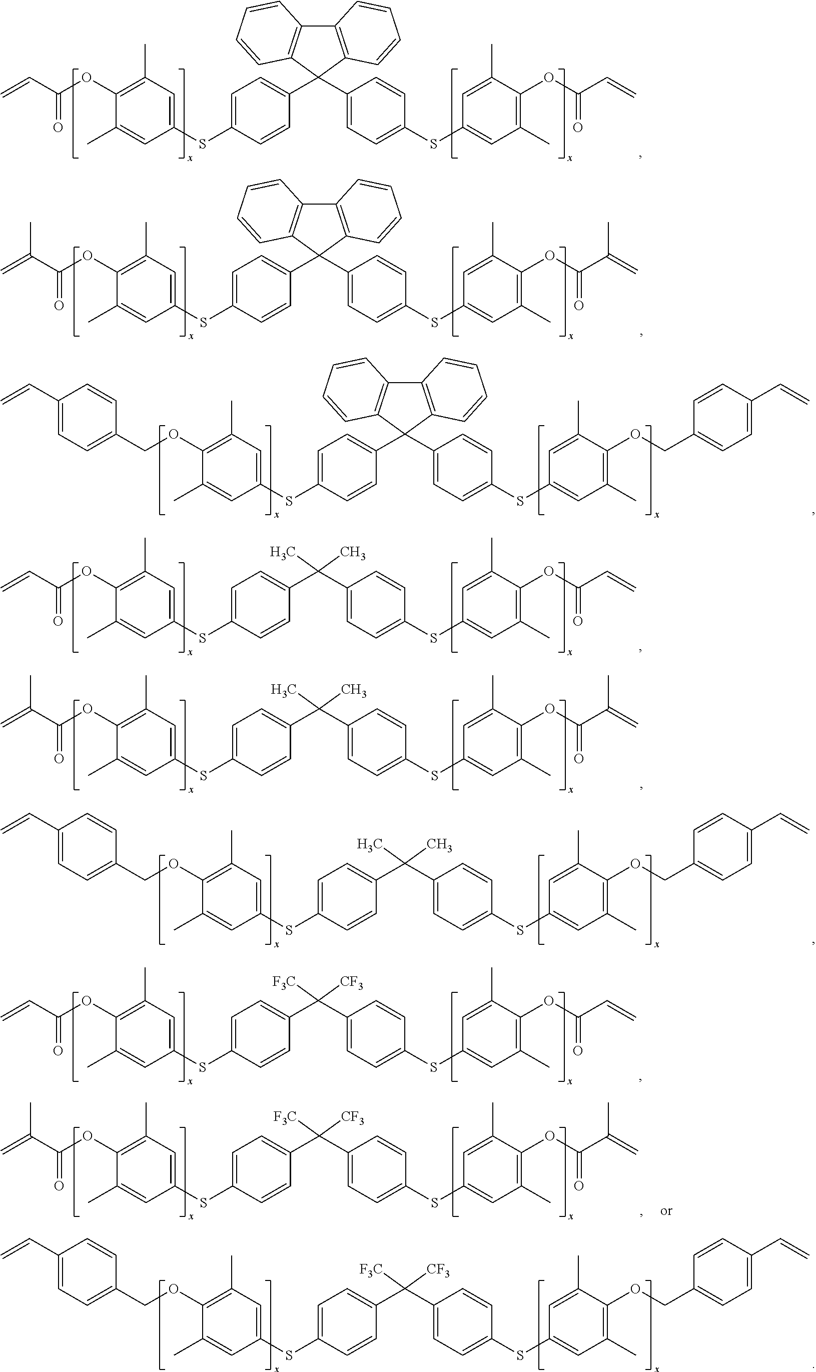

One embodiment of the disclosure provides a composition including 100 parts by weight of the described functionalized sulfur-containing polyphenylene oxide; and 10 parts to 50 parts by weight of a bismaleimide resin. If the bismaleimide resin amount is too low, the curing will be incomplete, and the cured composition will have an insufficient mechanical strength and a poor thermal stability. If the bismaleimide resin amount is too high, the cured composition will be hard and brittle (even cause the resin precipitate).

In some embodiments, the composition further includes 1 to 50 parts by weight of a functionalized polyphenylene oxide having a chemical structure of

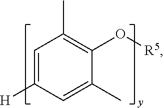

wherein R⁵ is

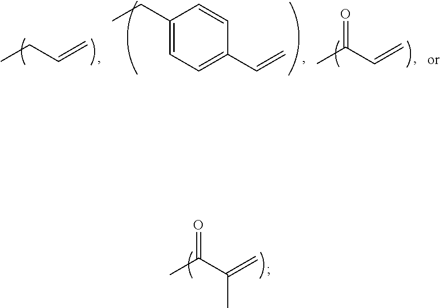

and y is an integer of 1 to 60. In some embodiments, y is an integer of 1 to 20. The functionalized polyphenylene oxide may further includes the mechanic strength of the cured composition. If the functionalized polyphenylene oxide amount is too high, the dielectric properties of the cured composition will be affected.

In some embodiments, the composition further includes 1 to 5 parts by weight of a radical initiator, 10 to 70 parts by weight of an inorganic filler, and 30 to 60 parts by weight of a solvent. If the inorganic filler amount is too low, the coefficient of thermal expansion of the cured composition will be too high, and the dimensional stability will be insufficient. If the inorganic filler amount is too high, the flowability of the composition will be insufficient, and the cured composition will be hard and brittle. If the solvent amount is too low, the flowability of the composition will be insufficient, and the resin will be unevenly dispersed. If the solvent amount is too high, the drying and curing period will be too long (even leave residual solvent).

In some embodiments, the inorganic filler includes glass, quartz, ceramic, or a combination thereof. In some embodiments, the solvent includes toluene, xylene, butanone, or N-methyl pyrrolidone. In some embodiments, the radical initiator includes azobisisobutyronitrile (AIBN), benzoyl peroxide (BPO), ammonium persulfate, or the like.

One embodiment of the disclosure provides a film formed by curing the described composition. For example, glass fiber cloths can be impregnated in the composition, then stacked between copper foils, and then heated and laminated to form a copper clad laminate. On the other hand, the composition can be used to form the film by any method. For example, the composition can be spray coated, blade coated, mold shaped, or shaped by another method, and then cured (e.g., dried) to obtain the film.

It should be understood that the sulfur-containing compound can be used to form the sulfur-containing polyphenylene oxide but is not limited thereto. The sulfur-containing polyphenylene oxide can be used to form the functionalized sulfur-containing polyphenylene oxide but is not limited thereto. The functionalized sulfur-containing polyphenylene oxide can be used to form the composition but is not limited thereto. The composition can be used to form the film but is not limited thereto. The sulfur-containing compound, the sulfur-containing polyphenylene oxide, the functionalized sulfur-containing polyphenylene oxide, and the composition can be individually applied in any available field.

Below, exemplary embodiments will be described in detail so as to be easily realized by a person having ordinary knowledge in the art. The inventive concept may be embodied in various forms without being limited to the exemplary embodiments set forth herein. Descriptions of well-known parts are omitted for clarity.

### EXAMPLES

### Example 1

Fluorenone (93.7 g, 520 mmol), thioanisole (258 g, 2080 mol), and 3-mercaptopropionic acid (5 g, 52 mmol) were dissolved in trifluoroacetic acid (400 g), and then heated to 60° C. to react for 24 hours under nitrogen. The reaction result was cooled, and dichloromethane and water were added thereto several times to extract the organic phase. The organic phase was collected, and solvent thereof was removed to obtain a solid precipitate product. The reaction is shown below:

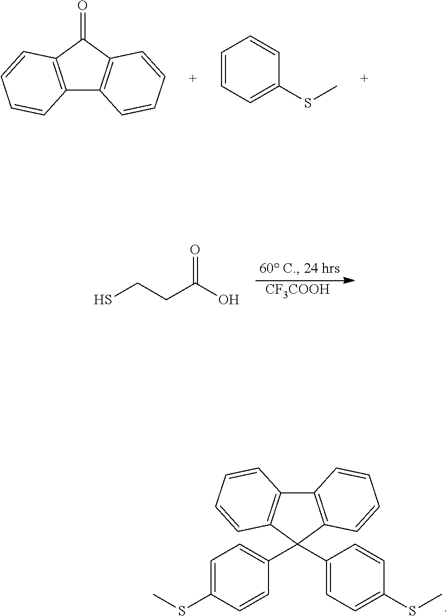

The above product (82 g, 200 mmol) was placed into a mixed solvent of dichloromethane (400 mL) and acetonitrile (50 mL) in ice bath (0° C.) under nitrogen. Concentrated nitric acid (72 g) was dropwise added to the above mixture. After the dropwise addition was completed, the mixture was returned to 25° C. to react for 4 hours. The reaction result was poured into an aqueous solution of NaOH, and then stirred for 1 hour. The above mixture was extracted by dichloromethane several times. The organic phase was collected, and solvent thereof was removed. The concentrate was washed by ethyl acetate several times and then dried to obtain a sulfur-containing compound S1 (81 g, yield was about 92%). The ¹H NMR spectrum (400 MHz, CDCl₃) of the sulfur-containing compound S1 is shown below: δ2.73 (s, 6H), 7.34-7.36 (m, 8H), 7.41-7.45 (t, 2H), 7.53-7.55 (d, 4H), 7.81-7.83 (d, 2H). The reaction is shown below:

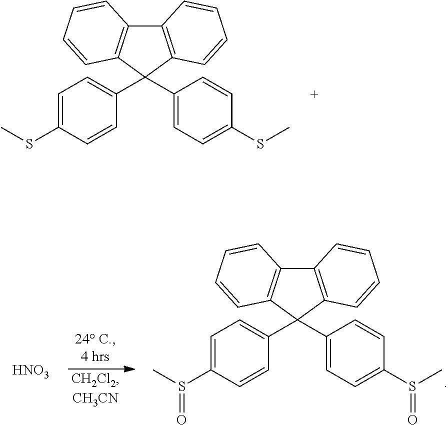

### Example 2

The sulfur-containing compound S1 in Example 1 (0.5 g, 1.25 mmol) and polyphenylene oxide oligomer (4.7 g, 2.5 mmol, Mn=1584, Mw=3524, PDI=2.22, synthesized according to CN10872379A) were dissolved in dichloromethane (100 mL) at 15° C. Eaton's reagent (25 mL) was slowly and dropwise added to the solution to react at 12° C. for 24 hours. The reaction result was slowly poured to de-ionized water at 10° C., and then extracted by dichloromethane several times. The organic phase was collected, and solvent thereof was removed to obtain a solid product.

The solid product was added into dimethylacetamide (DMAc, 100 mL) and heated to reflux for 6 hours. The reaction result was poured into de-ionized water, and then filtered to collect the filtered cake. The filtered cake was dried in a vacuum oven (50° C.) for 6 hours to obtain a sulfur-containing polyphenylene oxide P1. The sulfur-containing polyphenylene oxide P1 was analyzed by GPC to measure its molecular weight (Mn=4343, Mw=6027, and PDI=1.38) according to the standard ASTM D3593. The ¹H-NMR spectrum (400 MHz, CDCl₃) of the sulfur-containing polyphenylene oxide P1 is shown below: δ2.06 (s, 1H), 2.13 (s, 6H), 2.19 (s, 1H), 6.37 (s, 0.4H), 6.46 (s, 0.4H), 6.48 (s, 2H), 7.09-7.13 (m, 0.6H), 7.36 (m, 0.2H), 7.76-7.78 (d, 0.1H). The reaction is shown below:

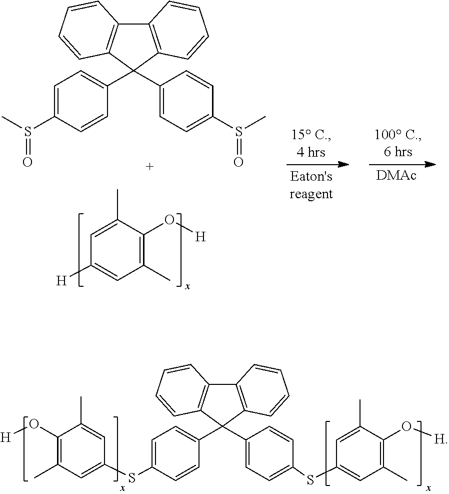

In the above formula, x is 2 to 22.

### Example 3

The sulfur-containing polyphenylene oxide P1 in Example 2 (5 g, 1.25 mmol), methacrylic anhydride (3.3 g, 21.5 mmol), sodium acetate (0.01 g, 1.25 mmol), and DMAc (50 mL) were evenly stirred and then heated to 75° C. to react for 2 hours. Subsequently, 500 mL of saturated saline solution was added to the reaction result to precipitate a solid. The above mixture was filtered to collect a filtered cake. The filtered cake was dried in a vacuum oven at 80° C. for 2 hours, thereby obtaining a functionalized sulfur-containing polyphenylene oxide Pla. The ¹H-NMR spectrum (400 MHz, CDCl₃) of the functionalized sulfur-containing polyphenylene oxide Pla is shown below: δ2.10 (s, 1H), 2.15 (s, 6H), 2.18 (s, 1H), 5.75 (s, 0.1H), 6.46 (s, 0.4H), 6.48 (s, 2H), 7.08-7.11 (m, 0.6H), 7.36 (m, 0.2H), 7.76-7.78 (d, 0.1H). The functionalized sulfur-containing polyphenylene oxide Pla was analyzed by GPC to measure its molecular weight (Mn=4377, Mw=6030, and PDI=1.45). The reaction is shown below:

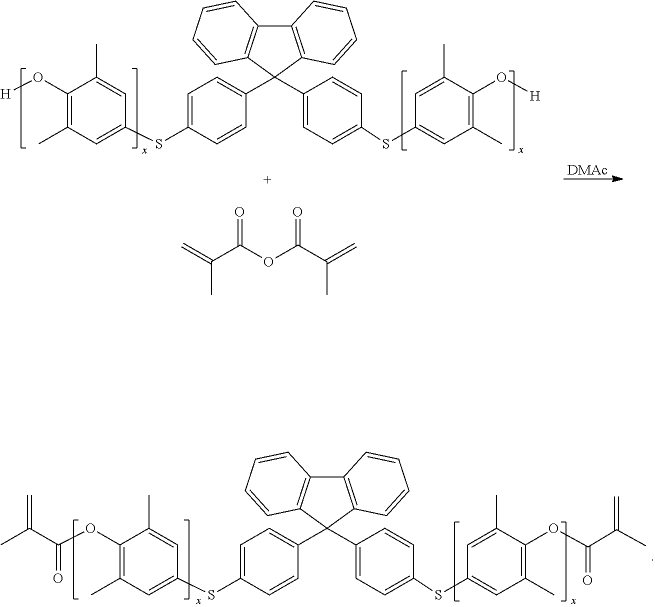

In the above formula, x is 2 to 22.

### Example 4

The sulfur-containing compound S1 in Example 1 (5 g, 12.5 mmol) and 2,6-dimethylphenol (3 g, 25 mmol) were dissolved in dichloromethane (50 mL) at 15° C. Eaton's reagent (50 mL) was slowly and dropwise added to the solution to react at 12° C. for 24 hours. The reaction result was slowly poured to de-ionized water at 10° C., and then extracted by dichloromethane several times. The organic phase was collected, and solvent thereof was removed to obtain a solid product.

The solid product was added into dimethylacetamide (DMAc, 100 mL) and heated to reflux for 6 hours. The reaction result was poured into de-ionized water, and then filtered to collect the filtered cake. The filtered cake was dried in a vacuum oven (50° C.) for 6 hours to obtain a sulfur-containing polyphenylene oxide P2. The ¹H-NMR spectrum (400 MHz, CDCl₃) of the sulfur-containing polyphenylene oxide P2 is shown below: δ2.21 (s, 12H), 6.94-6.96 (d, 4H), 7.02-7.04 (d, 4H), 7.13 (s, 4H), 7.22-7.26 (m, 2H), 7.32-7.34 (m, 4H), 7.72-7.74 (d, 2H). The reaction is shown below:

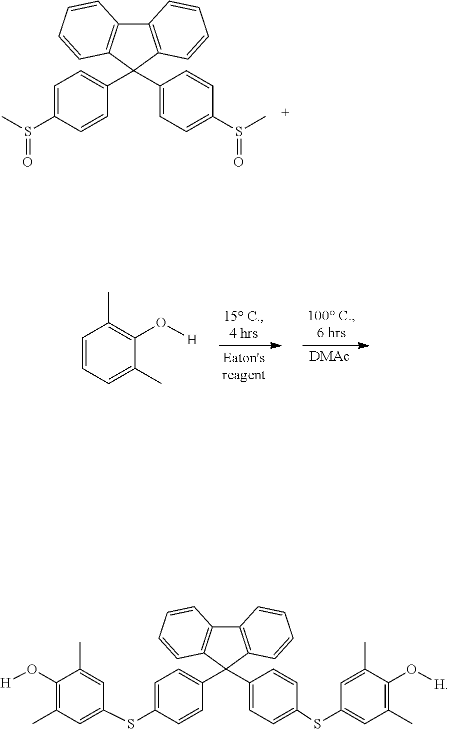

### Example 5

The sulfur-containing polyphenylene oxide P2 in Example 4 (2 g, 27.3 mmol), methacrylic anhydride (8.4 g, 54.7 mmol), sodium acetate (0.2 g, 25 mmol), and DMAc (20 mL) were evenly stirred and then heated to 75° C. to react for 2 hours. Subsequently, 500 mL of saturated saline solution was added to the reaction result to precipitate a solid. The above mixture was filtered to collect a filtered cake. The filtered cake was dried in a vacuum oven at 80° C. for 2 hours, thereby obtaining a functionalized sulfur-containing polyphenylene oxide P2a. The ¹H-NMR spectrum (400 MHz, CDCl₃) of the functionalized sulfur-containing polyphenylene oxide P2a is shown below: δ2.09 (s, 6H), 2.10 (s, 12H), 6.38 (s, 2H), 7.10 (s, 8H), 7.13 (s, 4H), 7.29-7.35 (m, 2H), 7.35-7.38 (m, 4H), 7.75-7.77 (d, 2H). The reaction is shown below:

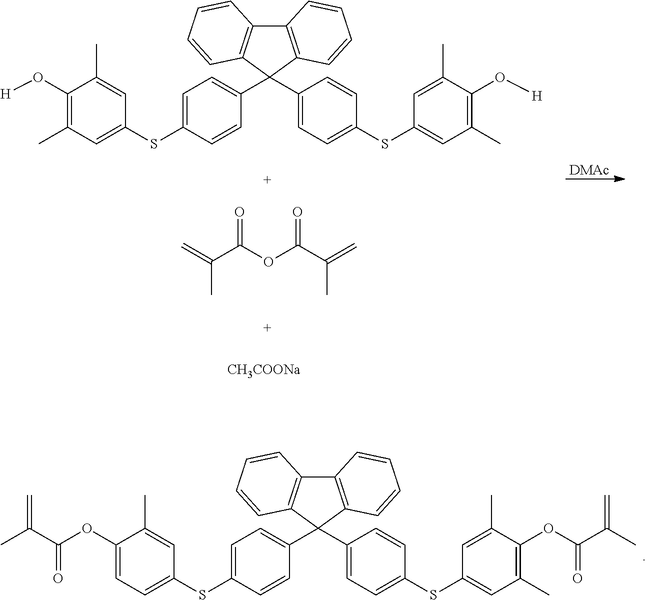

### Comparative Example 1

SA90 commercially available from Sabic was analyzed to measure its Mn (according to the standard ASTM D3593), Tg (according to the standard ASTM D3418), Td (5 wt %, according to the standard ASTM E2550), and solubility in toluene and xylene (40 wt %). SA90 had a chemical structure

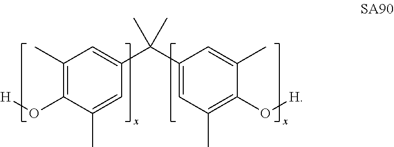

had Mn of 2200, Tg of 156° C., Td (5 wt %) of 402° C., and an excellent solubility in toluene and xylene.

### Example 6

The sulfur-containing polyphenylene oxide P1 in Example 2 had Mn of 4343, Tg of 180° C., Td (5 wt %) of 422° C., and an excellent solubility in toluene and xylene. As shown in Comparative Example 1 and Example 6, the sulfur-containing polyphenylene oxide P1 had a better thermal stability than that of SA90.

### Comparative Example 2

SA9000 commercially available from Sabic was used to form a dielectric plate to measure its dielectric constant at 10 kHz (according to the standard ASTM D150), dielectric loss at 10 kHz (according to the standard ASTM D150), and coefficient of thermal expansion at 150° C. (according to the standard ASTM E831). SA9000 had a chemical structure

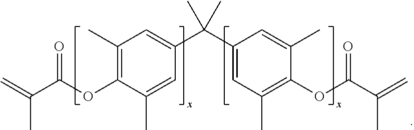

The phosphorous-containing polyphenylene oxide from Chang Chum Group (hereinafter referred as Chang Chun phosphorous-containing polyphenylene oxide) in TWI537281B was used to form a plate to measured its properties. PQ-60 commercially available from CHIN YEE CHEMICAL INDUSTRIES CO., LTD (hereinafter referred as PQ-60) in TWI678390B was used to form a plate to measure its properties. Chang Chun phosphorous-containing polyphenylene oxide had a chemical structure

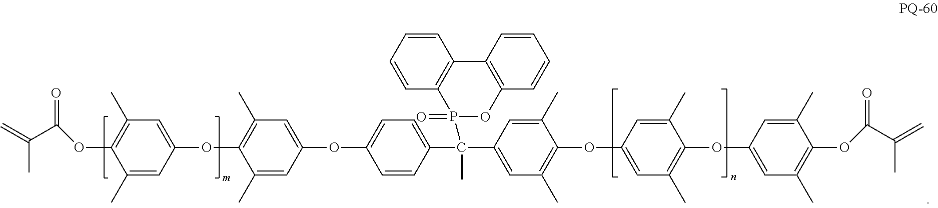

had a chemical structure

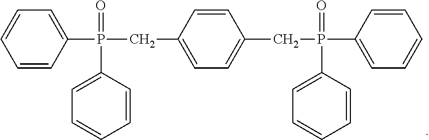 [SMILES: O=P(Cc1ccc(CP(=O)(c2ccccc2)c2ccccc2)cc1)(c1ccccc1)c1ccccc1]

The plate formed from SA9000 had a dielectric constant at 10 kHz of 2.52, a dielectric loss at 10 kHz of 0.0036, and a coefficient of thermal expansion of 47.8 ppm. The plate formed from Chang Chun phosphorous-containing polyphenylene oxide had a dielectric constant at 10 kHz of 2.78, a dielectric loss at 10 kHz of 0.0060, and a coefficient of thermal expansion of 48.0 ppm. The plate formed from PQ-60 had a dielectric constant at 10 kHz of 3.81 and a dielectric loss at 10 kHz of 0.0044.

### Example 7

The functionalized sulfur-containing polyphenylene oxide Pla and a bismaleimide resin (BMI commercially available from K·I chemical) having a weight ratio of about 100:15 were mixed and then cured to form a plate, which had a dielectric constant at 10 kHz of 2.72, a dielectric loss at 10 kHz of 0.0033, and a coefficient of thermal expansion of 34.8 ppm. Compared to the plates in Comparative Example 2, the plate in Example 7 had better dielectric properties and dimensional stability.

It will be apparent to those skilled in the art that various modifications and variations can be made to the disclosed methods and materials. It is intended that the specification and examples be considered as exemplary only, with the true scope of the disclosure being indicated by the following claims and their equivalents.

What is claimed is:

1. A sulfur-containing compound, having a chemical structure of 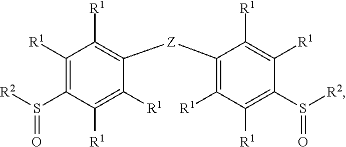
wherein each R¹ is independently H or F;
each R² is independently C₁₋₁₀ alkyl group or an aryl group substituted with a C₁₋₁₀ alkyl group; and
Z is 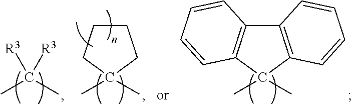
each R³ is independently H, F, C₁₋₆ linear alkyl group, C₁₋₆ fluorinated linear alkyl group, C₃₋₆ cycloalkyl group, C₃₋₆ fluorinated cycloalkyl group, C₃₋₆ branched alkyl group, or C₃₋₆ fluorinated branched alkyl group, and
n is an integer of 0 to 10.

2. The sulfur-containing compound as claimed in claim 1, having a chemical structure of

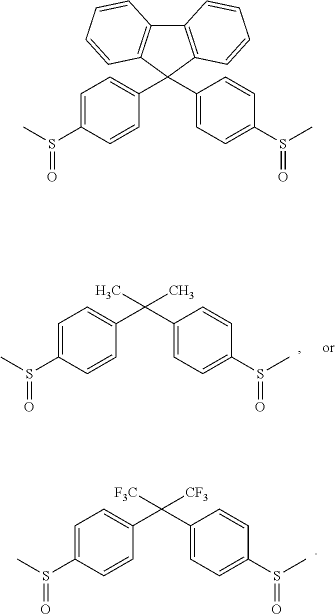

3. A sulfur-containing polyphenylene oxide, having a chemical structure of 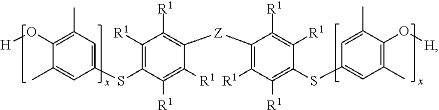
wherein each R¹ is independently H or F;
Z is 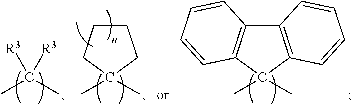
each R³ is independently H, F, C₁₋₆ linear alkyl group, C₁₋₆ fluorinated linear alkyl group, C₃₋₆ cycloalkyl group, C₃₋₆ fluorinated cycloalkyl group, C₃₋₆ branched alkyl group, or C₃₋₆ fluorinated branched alkyl group, and
n is an integer of 0 to 10; and
each x is an independent integer of 1 to 60.

4. The sulfur-containing polyphenylene oxide as claimed in claim 3, having a chemical structure of

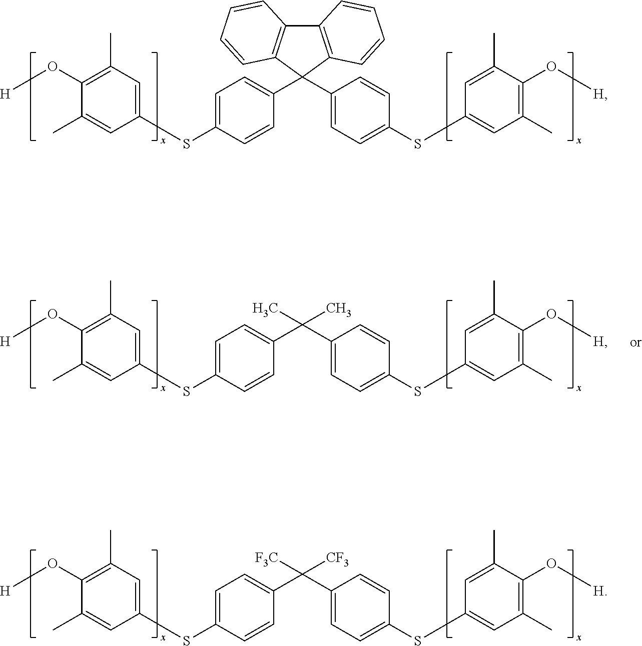

5. A functionalized sulfur-containing polyphenylene oxide, having a chemical structure of 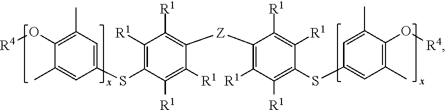
wherein each R¹ is independently H or F;
Z is 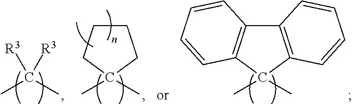
each R³ is independently H, F, C₁₋₆ linear alkyl group, C₁₋₆ fluorinated linear alkyl group, C₃₋₆ cycloalkyl group, C₃₋₆ fluorinated cycloalkyl group, C₃₋₆ branched alkyl group, or C₃₋₆ fluorinated branched alkyl group, and
n is an integer of 0 to 10,
each R⁴ is independently 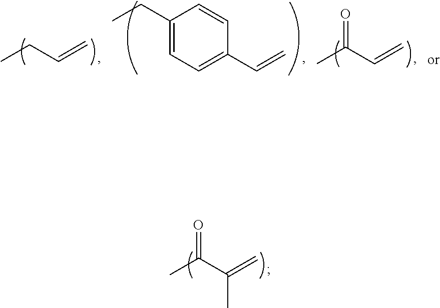
and
each x is an independent integer of 1 to 60.

6. The functionalized sulfur-containing polyphenylene oxide as claimed in claim 5, having a chemical structure of 2

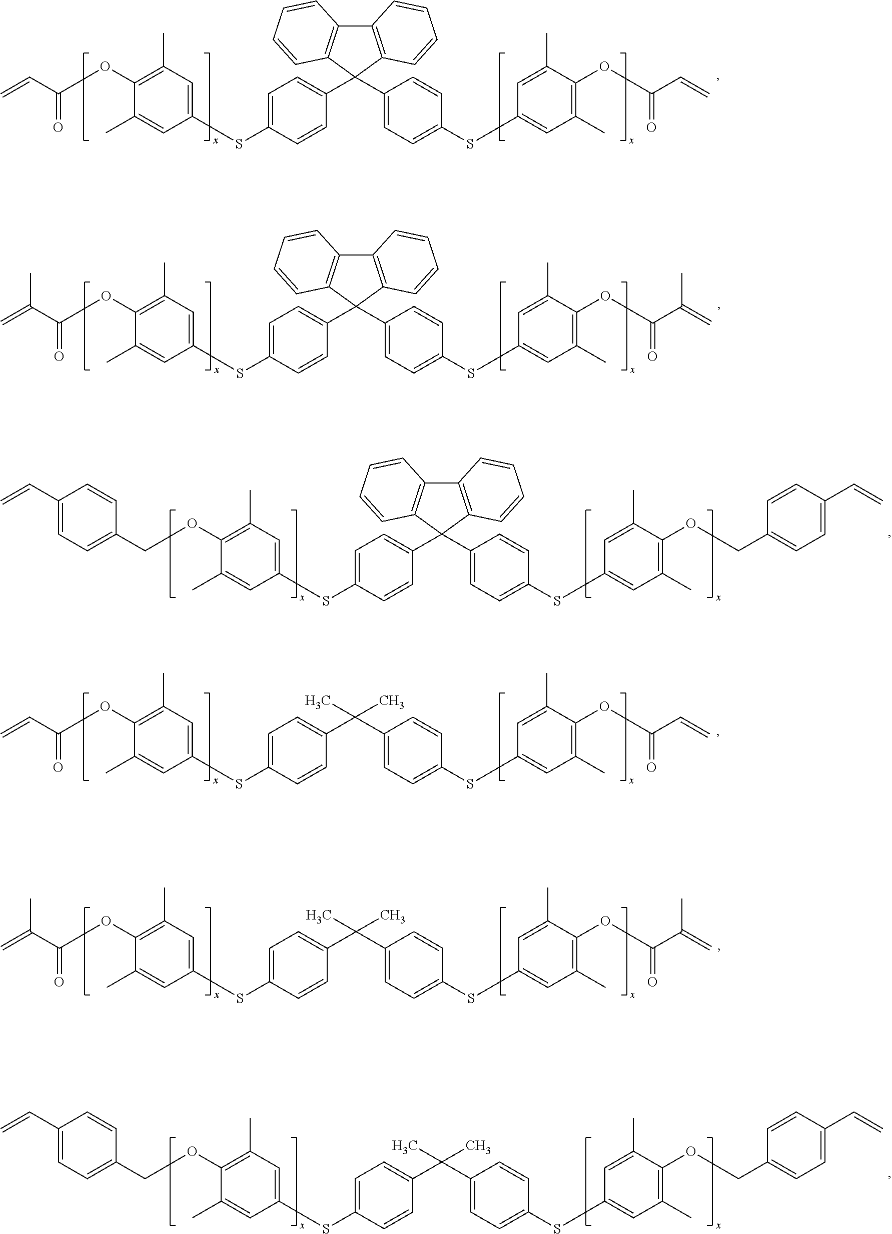

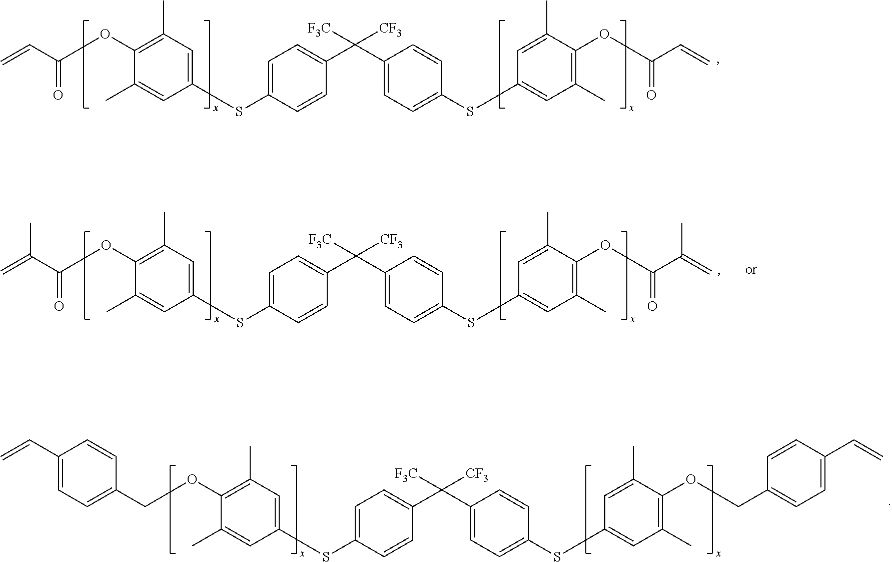

7. A composition, comprising:
100 parts by weight of the functionalized sulfur-containing polyphenylene oxide as claimed in claim 5; and
10 parts to 50 parts by weight of a bismaleimide resin.

8. The composition as claimed in claim 7, further comprising 1 to 50 parts by weight of a functionalized polyphenylene oxide having a chemical structure of 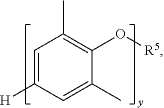
wherein R⁵ is 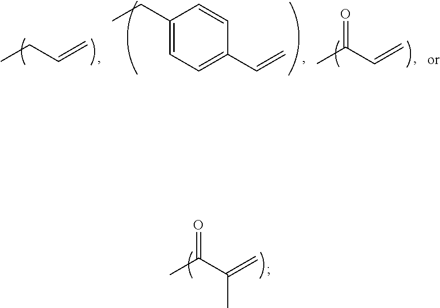
and
y is an integer of 1 to 60.

9. A film, being formed by curing the composition as claimed in claim 7.
